(19) United States
(12) Patent Application Publication
(10) Pub. No.: US 2013/0071435 A1
(43) Pub. Date: Mar. 21, 2013

(54) NOVEL HERBAL FORMULATION FOR THE MODULATION OF IMMUNE SYSTEM OF HIV INFECTED PATIENTS AND A PROCESS OF PREPARATION THEREOF

(71) Applicants: Govind Prasad Dubey, Varanasi (IN); Aruna Agrawal, Varanasi (IN); Nirupama Dubey, Kattankulathur (IN); Shipra Dubey, Kattankulathur (IN); Sunil Kumar Swamy Rajan, Bangalore (IN); Arun Kumar Swamy Rajan, Bangalore (IN); Vedant Arun, Bangalore (IN)

(72) Inventors: Govind Prasad Dubey, Varanasi (IN); Aruna Agrawal, Varanasi (IN); Nirupama Dubey, Kattankulathur (IN); Shipra Dubey, Kattankulathur (IN); Sunil Kumar Swamy Rajan, Bangalore (IN); Arun Kumar Swamy Rajan, Bangalore (IN); Vedant Arun, Bangalore (IN)

(73) Assignee: Sunil Kumar Swamy Rajan and Arun Kumar Swamy Rajan, Bangalore (IN)

(21) Appl. No.: 13/465,656

(22) Filed: May 7, 2012

(30) Foreign Application Priority Data
Sep. 20, 2011 (IN) 3235/CHE/2011

(51) Int. Cl. A61K 36/81; A61K 36/53; A61K 36/899; A61P 37/02; A61P 31/18; A61P 25/00; A61P 25/22; A61P 25/24; A61P 13/12; A61P 1/16; A61P 29/00; A61P 9/00; A61P 7/00; A61P 1/12; A61P 3/04; A61P 11/14; A61P 39/06; A61P 25/28; A61K 36/185

(52) U.S. Cl. 424/278.1

(57) ABSTRACT

An herbal formulation for the modulation of immune system of HIV infected patients and a process of preparation thereof are provided. The process includes (i) preparing a hydromethanolic extract of at least one plant selected from Hippophae rhamnoides, Convolvulus pluricaulis, Withania somnifera, Ocimum sanctum, and Cynodon dactylon at 80-90° C., maintaining the pH of the solution between 6-7; (ii) separating the active compound chromatographically; and (iii) subjecting the active compounds to the step of molecular characterization.

### FIELD OF INVENTION

The present invention relates to a herbal formulation for the modulation of immune system of HIV infected patients and a process of preparation thereof for modulation of immune system among HIV infected patients and also for the regulation of immune profile against various bacterial, viral and fungal infections among HIV patients in order to enhance general body resistance against HIV infection during the latency period of disease condition varying from 2-10 years wherein the patients develop complications due to decline of immunity.

### BACKGROUND OF INVENTION

The human immunodeficiency virus (HIV) infection has attracted maximum attention of the scientific community with an associated interest in finding remedial measures for the prevention and management of acquired immuno-deficiency syndrome (AIDS). The recent advancements in basic immunology aided the scientific community to examine functioning of the immune system and the causes of its failure. The development of monoclonal antibody technology led to the characterization of large number of surface receptor molecules on leukocytes which permit tracking disease progression. HIV (AIDS) is one of the most important clinical challenges for the medical sciences in terms of prevention and management.

The challenges encountered due to the viral infection are manifold involving social, medical and ethical aspects. AIDS is a real global pandemic with infections reported from every corner of the world. In the absence of the affordable and accessible diagnostic facilities, the real global viral incidence is not known. In 2001, 40 million people were living with HIV/AIDS worldwide, prevalence rate was 1.2%, about 5 million became infected and nearly 3 million people died of the infection. The high rates of fatality together with the lack of suitable treatment or availability of an effective vaccine collectively compromised the quality of life making HIV/AIDS a serious global health problem.

It is an established fact that the human immunodeficiency virus (HIV) infected individuals gradually show declining trend of both cellular and humeral immunity, deteriorating various functions of vital organs particularly reticulo-endothelial system, nervous system and kidney function

The main cause of immune defect in AIDS is the dysfunction of the thymus-derived lymphocytes (T-cells), characterized by the presence of the CD4 surface molecules which are the cellular receptors for HIV.

AIDS is characterized by cellular immunodeficiency in the infected subjects. The time from infection to onset of the disease progression is approximately 8-10 years.

HIV that belongs to the family of human retrovirus and the subfamily of Lenti-viruses is the etiologic agent of AIDS. HIV is transmitted by sexual contact, blood, blood products or by infected mother to infant. In the initial period, the patients are asymptomatic. In due course, depending on immunity of the individual subject and severity of the infection, a syndrome of clinical symptoms are manifested including infections like tuberculosis, Cryptococcus, pneumonia, etc. In the latent period of the viral infection, functional abnormalities of the lymphocytes are manifested. As a result of wide variation in host factors including HLA and differential anti-viral immune responses, the clinical manifestation and the rate of disease progression too vary significantly.

Psycho-neuro-endocrine manifestation due to HIV infection is common among the individuals. In course of disease progression of HIV infection could cause peripheral neuropathy and sub-acute encephalitis including dementia complex. Clinically sub-acute encephalitis is characterized by poor memory, inability to concentrate, apathy and psychomotor retardation, focal motor abnormalities and behavioral changes.

The currently available anti-retroviral therapy (ART) has a limited role in increasing life expectancy and improving the quality of life in seropositive subjects. The heavy costs associated with anti-retroviral therapy, and unacceptable levels of toxic side effects, the therapy could not receive wider acceptance by the medical world. In the developing countries, reverse transcriptase inhibitors that inhibit the in vivo proliferation of spread of infectious virus have been in wide use. In the absence of effective cure, immunorestorative therapy seems to be a better remedial measure. It is therefore justified to propose a polyherbal medicine that is well tolerated and free from side effects.

Rasayana therapy is one of the major clinical disciplines in Ayurvedic system of medicine. The main object of rasayana therapy is to promote general body immunity which is helpful in prevention of disease and early decay of the body. In all classical Ayurvedic texts several drugs are prescribed showing rasayana property. The rasayana is not specific treatment for disease conditions rather it has a restorative property capable of preventing the progression of illness by increasing the body resistance. Taking lead from ancient wisdom we have screened large number of medicinal plants and selected five plants showing rasayana property to prevent the progression of T-lymphocytes destruction caused by HIV infection.

Hence there felt a need to provide an herbal formulation for the prevention and management of abnormal immune profile in HIV infected patients with the following objectives

### OBJECTS OF INVENTION

The main object of our invention is to increase the general body immunity ie CD4, CD8 cell count, IgG, IgM, IgA, general blood picture (TC, neurophils, lymphocytes, eosinophil, hemoglobin and total serum protein) including inflammatory cytokines in HIV infected patients in order to enhance general body resistance against HIV infection.

Another object of this invention is to propose a plant based formulation useful in stimulating the immune profile among HIV infected patients with the view to prevent opportunistic infection particularly tuberculosis and pneumonia and to enhance longevity of HIV patients.

Yet another object of this invention is to propose a plant based therapy that can increase humoral and cellular immunity and thus can prevent the early decline of CD4 cell count.

A further object of this invention is to propose a novel therapeutic intervention which can act as anti-HIV-1 protease inhibitors.

An additional object of this invention is to propose a new therapeutic intervention that can slow down the process of thrombocytopenia and neuropenia.

Further additional object of this invention is to protect the early onset of cognitive decline among HIV patients as loss of hippocampal neurons is noticed in HIV patients causing dementia.

Still object is to protect the liver function and renal function in HIV patient's manifestation due to secondary infection.

The another object of this invention is to manage the anxiety and depression among HVI patients which is markedly associated with these patients.

### STATEMENT OF INVENTION

According to this invention, there is provided a novel herbal formulation for the modulation of immune system of HIV infected patients and a process of preparation thereof, comprising (i) preparing a hydromethanolic extract of at least one plant selected from Hippophae rhamnoides, Convolvulus pluricaulis, Withania somnifera, Ocimum sanctum, and Cynodon dactylon at 80-90° C., maintaining the pH of the solution between 6-7 (ii) separating the active compound chromatographically and (iii) subjecting the active compounds to the step of molecular characterization.

Further, according to this invention there is provided a process for the preparation of novel plant based Ayurvedic formulation as claimed in claim-I comprising of preparing aqueous adding methanolic extract of Hippophae rhamnoides (Badriphal, Fruits), Convolvulus pluricaulis (Shankhapushpi, whole plant), Withania somnifera (Ashwagandha; Root), Cynodon dactylon (Durva, Whole plant) and Ocimum sanctum (Tulsi-Leaves), by using aqueous and methanol (50:50) at 80°-90° C. temperature and maintaining pH of solution between 6-7, separating the active compound chromatographically of each plant material (extract) by using TLC, HPLC and HPTLC separation of the molecules of plant extract by using GCMS, LCMS and 2D NMR.

### BRIEF DESCRIPTION OF THE FIGURES

FIG. 1 is a flow diagram of the process.

### DETAILED DESCRIPTION OF THE INVENTION

The hydro-methanolic extract of seven Ayurvedic plants i.e. Hippophae rhamnoides, Withania somnifera, Convolvulus pluricaulis, Cynodon dactylon, and Ocimum sanctum by using 50% water and 50% methanol was prepared for the development of present novel formulation by conducting various experimental and clinical studies. The water utilized for extraction was decontaminated for any type of bacterial or abnormal growth by using reverse osmosis plant. After completing extraction procedure the extracted materials was taken to determine the presence of percentage of active molecules in all the five selected plants and were identified by HPLC, HPTLC, GCMS, LCMS and 2DNMR procedures.

The biological activity was evaluated by conducting various experimental animal models of immune injury of single plant extract as well as combined formulation acting on various targets responsible for immune deficiency through different mode of actions.

The interaction between chemical constituents and biological markers like IgG, IgM, IgA, CD4, CD8, Total WBC count, neutrophils, lymphocytes, eosinophils and hemoglobin including inflammatory cytokines and oxidative stress markers were evaluated and role of drug was established through such studies.

Before utilizing the drug for human consumption the pre-clinical acute, sub-acute and chronic toxicity studies were carried out to determine the safety profile. Further, the efficacy profile of test formulation mainly immunomodulaotry activity, CD4 ameliorating and stabilizing effects were determined in animal studies. The mode of action of single and combined formulation was established through various mechanism based studies. Similarly the effective dose of each plant extract material was determined through action on different targets (bio-markers) involved with the disease condition.

### Extraction Procedure:

The dried fruits of Hippophae rhamnoides, whole plant of Convolvulus pluricaulis, root of Withania somnifera whole plant of Cynodon dactylon, and leaves of Ocimum sanctum were utilized to obtain extracted material of the plants. The water and methanol 50:50 ratio was utilized for the extraction. After extraction the extracted material was taken for identification and separation of active compound present in the extract of the plants by using TLC, HPLC, HPTLC, GCMS, and LCMS. Afterwards the molecular characterization was carried out by using IR and NMR.

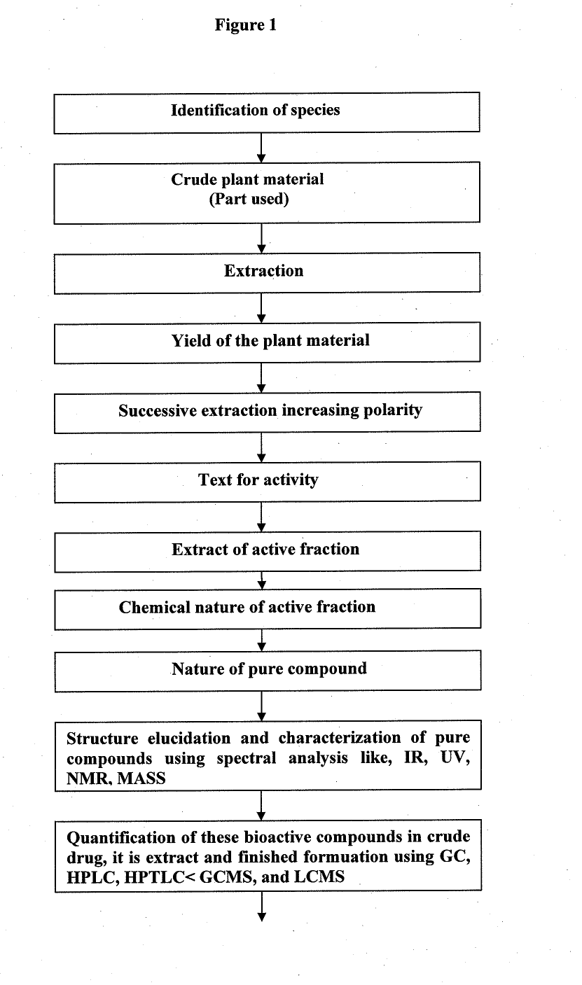

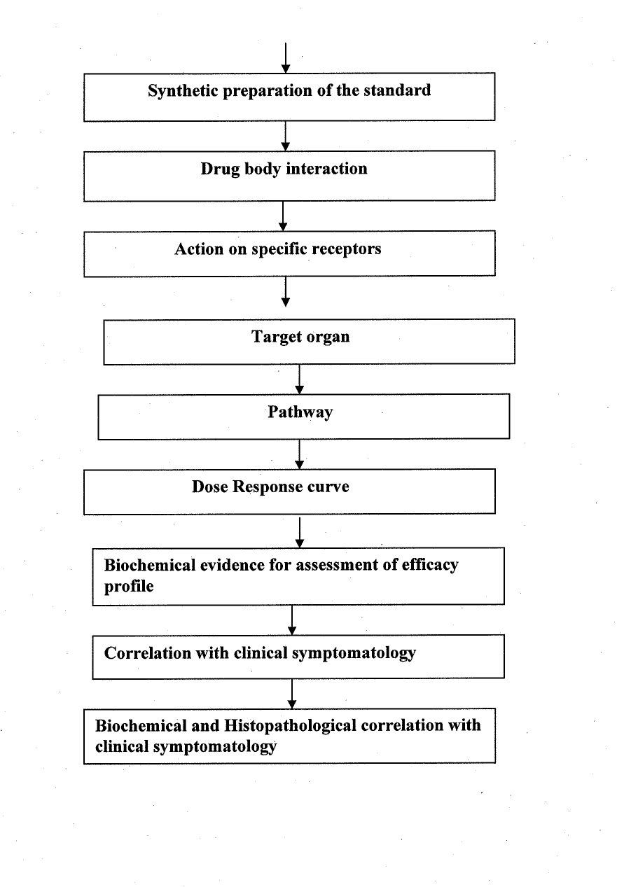

The extraction was done at the temperature of 80-90° C. The pH of the solution was maintained between 6-7. The steps which were adopted and carried out to isolate the active compound, preparation of test drug as well as to develop a final new drug have been illustrated in FIG. 1.

According to this invention there is provided an Ayurvedic formulation for the prevention and management of deficiency of immune profile among diagnosed HIV infected patients, with the object to improve their quality of life, to prolong the longevity and to prevent them from opportunistic infections particularly tuberculosis and pneumonia. The present test formulation comprising of the following five ingredients—

| Name of the plants | Part used |
|---|---|
| 1. Hippophae rhamnoides | Fruits |
| 2. Withania somnifera | Root |
| 3. Convolvulus pluricaulis | Whole plant |
| 4. Cynodon dactylon | Whole plant |
| 5. Ocimum sanctum | Leaves |

Preferably the aforesaid plants are present in the test drug in the following range of doses—

| Name of the plants | Dose |
|---|---|
| 1. Hippophae rhamnoides | 150-400 mg/day |
| 2. Withania somnifera | 100-400 mg/day |
| 3. Convolvulus pluricaulis | 100-300 mg/day |
| 4. Cynodon dactylon | 125-400 mg/day |
| 5. Ocimum sanctum | 75-300 mg/day |

The formulation may also comprise known additives such as minerals, vitamins, salts filler (for capsulation or to prepare syrup) and binders, if required to present in trace amount.

Thus any known additive or supplement is added to prepare the final formulation as required and present in trace amount. Reference is made here in capsule form. However, it would be apparent that the preparation may also be in the form of syrup/tablet.

Preferably but without implying any limitation the preparation (formulation) comprises:

| Name of the plants | Dose |
|---|---|
| 1. Hippophae rhamnoides | 250 mg/day |
| 2. Withania somnifera | 150 mg/day |
| 3. Convolvulus pluricaulis | 175 mg/day |
| 4. Cynodon dactylon | 225 mg/day |
| 5. Ocimum sanctum | 150 mg/day |

The present Ayurvedic formulation is prepared out of five plant extracts namely Hippophae rhamnoides, Withania somnifera, Convolvulus pluricaulis, Cynodon dactylon, and Ocimum sanctum that are mixed in effective doses. The beneficial role of present test formulation is through its immunomodulatory activity as it enhances immunity against a pathogen by activating the immune system. HIV belongs to the Lentivirinae subfamily of retrovirus which has an RNA genome. The RNA genome is encapsulated with a core which in turn is enwrapped by an envelope. The virus gains entry to the target cells by binding to the CD4 receptor using the viral surface membrane glycoprotein 120. The CD4 receptor is present predominantly on T-helper lymphocytes which are the major target for the virus. Thus HIV principally infects CD4 helper T-lymphocytes. The cells are responsible for the initiation and maintenance of the immune responses to pathogens. Following the viral infection there is attrition in the CD4 cell population resulting in gradual dysfunction of the cellular immunity. Further, HIV also affects cells of the monocyte/macrophage lineage, and dendritic cells.

During the course of HIV infection there is gradual reduction in the number of CD4 cells and this phenomenon serves as a prognostic marker indicating the progression as well as classification of the disease state. CD4 cell count is also used to determine when the anti-retroviral or a microbial therapy should be instituted. The estimation of the serum immunoglobulin levels is a direct measure to detect the humoral immunity. Serum immunoglobulin refers to a group of serum molecules produced by plasma cells, they are soluble and counter the invasion of a pathogen. It has been demonstrated that the active constituents of the plant extracts could improve prognosis in the viral infection by means of immunomodulatory activities as well as anti-microbial, anti-inflammatory, anti-viral and anti-oxidant properties. Further, it is proposed that the present polyherbal formulation caused specific activation of T-lymphocytes, phagocytic cells as well as elevation in cytokine levels including gamma-interferon and tumor necrosis factor (TNF). Thus the mechanism of action of the present polyherbal formulation seems to be through activating the cell mediated immune system.

The humeral immunity is mediated by antibodies produced by plasma cells aided by CD4+ T-helper 2 (Th2) cells. Th2 activation and cytokine production, germinal centre formation and isotype switching affinity, maturation and memory cell generation come under T-help. Antibodies mediate pathogen or toxin neutralization, complement activation and opsonin promotion all contributing to pathogen elimination. The present polyherbal formulation has shown improvement in T-lymphocyte functioning and stalling of the kinetics of CD4+ cell reduction. Immune dysfunction leads to disease progression in HIV seropositive subjects. Through immunomodulatory properties, the polyherbal formulation enhanced the concentrations of IgG, IgM and IgA as well as the numbers of total white blood cells, neutrophils, and lymphocytes, further increased the levels of hemoglobin and total serum protein.

The viral infection may produce a variety of neurologic manifestations due to opportunistic infections. Monocyte macrophage lineage is predominantly infected. HIV infected individual may manifest both white matter legion as well as neuronal loss. A series of changes take place due to neurotoxicity of gp120, TNF-α, IL-1, IL-2, IL-6, TGF-β and endotheline. We concur that polyherbal formulation could delay the process of cell apoptosis in the neurons. The loss of cholinergic and glutamatergic receptors have shown that early action may prevent the deterioration of cognitive function due to prevention of decline in glutamatergic and cholinergic neurons.

During the latency period the protection of vital organs like brain, kidney and liver is essential. The structural damage of brain particularly limbic system can be prevented by the drugs which can increase the neural capability to combat T-cell and B-cell deficiency in HIV infected cases. The present test formulation has potentiality to fulfill above objects to a great extent.

### About the Plant:

1. Hippophae Rhamnoides:
   This is high altitude plant belongs to family Elaeagnaceae. Fruits and leaves have shown medicinal property. Hippophae rhamnoides is a rich source of flavonoids, vitamins, proteins, amino acids, folic acid, phytosterol, alpha-tocopherol and phenolic compounds. There are at least 24 chemical elements present in Seabuckthorn juice eg. nitrogen, phosphorous, iron, manganese, boron, calcium, aluminium, silicon and others. It has shown anti-oxidant, immuno-modulatory, anti-inflammatory and homocysteine lowering effects and uplifts the mental function.
2. Withania Somnifera:
   The plant belongs to family solanaceae, and is one of the ingredients of present test formulation. It has shown anti-stress, adoptogenic and hypotensive properties and is beneficial in the regulation of altered neurotransmitters through its active compound withanoloids, somniferine and withanine. One of the recent studies has indicated that Withania somnifera reconstruct the neuritic damage and also improves synaptic plasticity in the brain. Two lycowithanolides namely sitoindoside 1× (1) and sitoindoside ×(2) is isolated from Withania somnifera. This has shown a significant immuno-potentiating activity both in experimental as well as in in-vitro and in-vivo studies. This plant has been included due to potent anti-modulatory activity found useful in enhancing both cellular and humeral immunity.
3. Convolvulus Pluricaulis:
   It is one of the popular plants in Ayurvedic medicine, belongs to Convolvulaceae family. It is a perennial herb found throughout India in the plains area. Scopoletin, kaempferol-3-glucoside, kaempferol, 3,4-dihydroxycinnamic acid are the major active chemical constituents found in this plant. In addition it also contains β-Sitosterol-β-D-glucoside, glucose and an alkaloid ‘shankhapushpine’ showing pharmacological activity. This plant has shown Anti-anxiety, anti-depressant, hypotensive, immuno-modulatory, anti-oxidant and anti-inflammatory activity. It enhances mental competence. Studies have indicated that Convolvulus pluricaulis in combination with Withania somnifera has shown hepato-protective potential.
4. Cynodon Dactylon:
   It is a wild growth throughout India. It is a perennial creeping grass, rooting at every node, forming matted tufts. The whole plant contains sitosterol and carotene. Other compounds like vitamin C, cartone, palmitic acid, triterpenoides, alkaloids ergonovine and ergonovinine etc. are also present. Both HA litre DTH response indicated the Cynodon dactylon potentiates humoral as well as the cellular immunity. The augmentation of the humoral response was evidenced by an enhancement of antibody responsiveness to SRBC in mice of consequence of both pre and post Immunization protein treatment indicates the enhanced responsiveness of macrophages and B-lymphocytes, subsets involved in antibody syntheses.
5 Ocimum Sanctum:
   The plant belongs to family Lamiaceae, grows all over India up to 2000 meters height. It is grown in houses, temples and gardens. Some of the main chemical constituents of Tulsi are: Oleanolic acid, Ursolic acid, Rosmarinic acid, Eugenol, Carvacrol, Linalool, and β-caryophyllene. Aqueous extract from leaves showed both humeral and cell mediated immune-response in rats and mice, it is an immunomodulator.

### EXAMPLES

The invention will be illustrated but not limited by the following examples. Those skilled in the art recognize that various modifications can be made to the invention without departing from the spirit and scope thereof.

### Example-I

When the hydro-methanolic extract of Cynodon dactylon in the dose of 50 mg/kg, Ocimum sanctum 40 mg/kg, Convolvulus pluricaulis 45 mg/kg and Withania somnifera 50 mg/kg mixed and given to experimental animals showed activated T-cell response indicating an enhancement of antigenic potency and stimulated lymphocyte proliferative response. Thus the above combined formulation exerted cell mediated immune responsiveness.

### Example-II

When the hydro-methanolic extract of Convolvulus pluricaulis in the dose of 45 mg/kg, Withania somnifera 45 mg/kg and Hippophae rhamnoides 60 mg/kg mixed and given to immobilized stressed model animals the IgG, IgM and IgA immune responsive markers modulated in treated group than non-treatment group. A significant increase in the humeral immunity was noticed.

### Example-III

When the hydro-methanolic extract of Convolvulus pluricaulis in the dose of 50 mg/kg, Withania somnifera 35 mg/kg, Hippophae rhamnoides 45 mg/kg mixed and given to sleep deprivation stressed animals showing poor learning with deteriorated immunity, a reduced number of error in completing learning task were recorded in treated group of animals in comparison to non treatment control group where animals were induced only stress and no treatment. Thus the test drug has neuromodulatory potentials as it enhanced protein synthesis of neurons, modulated the cholinergic, glutamatergic, GABAergic, nor-adrenergic and dopaminergic receptors.

### Example-IV

After determination of safety and efficacy profile of single plant candidate as well as combined formulation in pre-clinical studies the formulation containing hydro-methanolic extract of selected ingredients in effective doses were evaluated in HIV infected patients for modulation of immune profile. When the hydro-methanolic extract of Hippophae rhamnoides in the dose of 325 mg/day, Ocimum sanctum 200 mg/day, Withania somnifera 250 mg/day and Cynodon dactylon 175 mg/day mixed and given to diagnosed HIV infected patients, improvement in decline in T-lymphocytes are observed. Further, reduced rate of decline of CD4 cell count and decrease in CD8 is also recorded. The continuous administration of drug indicated the stabilizing of CD4 cell count with decrease CD8 cell indicated arrest of progression of disease process.

### Example-V

When the hydro-methanolic extract of Hippophae rhamnoides in the dose of 325 mg/day, Ocimum sanctum 275 mg/day, Cynodon dactylon 325 mg/day mixed and given to HIV infected patients a significant increase in RBC, platelet count and hemoglobin level. The test formulation has shown potentiality to prevent neutropenia and thrombocytopenia in HIV infected patients. Thus the test formulation is an effective immuno-stimulant as IgG, IgM levels increased to a significant extent in treated group.

### Example-VI

When the hydro-methanolic extract of Hippophae rhamnoides 350 mg/day, Cynodon dactylon 225 mg/day and Ocimum sanctum in the dose of 375 mg/day mixed and given to HIV infected patients liver function and renal function improved and continuous application of the drug protected the HIV patients from the development of hepatitis and renal diseases particularly improved the micro-albuminuria, and reduced SGOT, SGPT along with alkaline phosphatase.

### Example-VII

The hydro-methanolic extract of Hippophae rhamnoides in the dose of 275 mg/day, Convolvulus pluricaulis 225 mg/day, Withania somnifera 325 mg/day mixed and given in the form of combined formulation to HIV patients, improvement in cognitive function including memory performance was observed. Improvement in depression level and sleep pattern of HIV patients was also determined.

### Example-VIII

The HIV infection causes persistence chronic inflammation. Severe oxidative stress has been reported in HIV infected patients. The hydro-methanolic extract of Hippophae rhamnoides in the dose of 350 mg/day, Withania somnifera 325 mg/day, Cynodon dactylon 225 mg/day mixed and given to HIV infected patients the elevated homocystein and inflammatory cytokines IL-₆ and TNF-α reduced significantly. The plant Hippophae rhamnoides is rich in containing folic acid, B₁, B₁₂ and other micronutrients therefore improved body resistance and feeling of well being is reported by HIV patients. It is proposed that the risk of CHD and neurodegenerative disorders was minimized by test formulation administration in HIV infected patients.

### Example-IX

The hydro-methanolic extract of Hippophae rhamnoides in the dose of 250 mg/day, Convolvulus pluricaulis 125 mg/day, Withania somnifera 175 mg/day, Cynodon dactylon 225 mg/day, Ocimum sanctum 150 mg/day mixed and administered to HIV infected patients showed increase of WBC and platelet count, T-lymphocyte cells count and hemoglobin also increased to a great extent, prevented neutropenia and thrombocytopenia. The most important result obtained out of the present clinical trial is the stabilization of cluster of differentiation (CD4), cell count. Further, the retarded level of IgG, IgM and IgA also enhanced. A neuromodulatory activity of test formulation is also reported as the present test drug checked the loss of cholinergic neurons, ameliorated the nor-adrenergic, GABAergic, dompaminergic pathways, particularly the gulatmatergic receptors. The drug also exerted anti-anxiety and anti-depression effects. The test formulation has potential role in the protection from kidney and liver diseases. As the test drug stabilized the loss of CD4 cell count the onset of opportunistic infections particularly tuberculosis, pneumonia including frequent cold and cough was prevented/the duration of onset was enhanced significantly, thus the longevity of HIV patients is increased and quality of life also improved.

Continuous use of test formulation did not show any adverse reaction even after its prolonged application.

Conventional treatment includes:
Becosule (1 capsule once in a day)
Fefol (1 capsule once in a day)
Bectrim DS (Once Tablet twice in a day)
Supplement of protein according to the serum protein level

The conventional treatment was given as per standard schedule. The test drug was given continuously. The long term follow-up study following test formulation treatment among HIV infected patients shows that the CD4 cell count following test formulation treatment considerably stabilized.

TABLE 1
Slowing down of the process of decline of CD4 cell count following test formulation treatment in HIV positive patients.

| | No. | | | | | | |
|---|---|---|---|---|---|---|---|
| Treatment | of | CD4 (cells/μL) | | | | | |
| group | cases | Initial | 1^{st} year | 2^{nd} year | 3^{rd} year | 4^{th} year | 5^{th} year |
| Conventional | 48 | 468.93 ± 131.78 | 407.88 ± 121.35 | 374.62 ± 93.22 | 323.05 ± 71.66 | 285.52 ± 54.03 | 236.84 ± 41.35 |
| treatment | | | | | | | |
| Conventional | 52 | 448.35 ± 129.52 | 419.75 ± 94.80 | 397.84 ± 63.77 | 384.97 ± 73.72 | 363.35 ± 59.85 | 344.88 ± 61.32 |
| treatment + | | | | | | | |
| Test | | | | | | | |
| formulation | | | | | | | |

TABLE 2
Table shows the less increase in elevation of CD8 following test drug treatment in HIV positive patients

| | No. | | | | | | |
|---|---|---|---|---|---|---|---|
| Treatment | of | CD8 (cells/μL) | | | | | |
| group | cases | Initial | 1^{st} year | 2^{nd} year | 3^{rd} year | 4^{th} year | 5^{th} year |
| Conventional | 48 | 536.93 ± 58.45 | 573.82 ± 63.04 | 728.32 ± 59.87 | 912.80 ± 79.45 | 988.52 ± 81.33 | 1008.34 ± 112.42 |
| treatment | | | | | | | |
| Conventional | 52 | 518.74 ± 61.36 | 543.42 ± 82.55 | 615.32 ± 73.75 | 644.73 ± 65.91 | 673.04 ± 82.04 | 712.42 ± 93.74 |
| treatment + | | | | | | | |
| Test | | | | | | | |
| formulation | | | | | | | |

TABLE 3
Effect of test formulation on immunologic markers among HIV infected patients

| | No. | | | | | | |
|---|---|---|---|---|---|---|---|
| Treatment | of | lg (mh/d1) | | | | | |
| group | cases | Initial | 1^{st} year | 2^{nd} year | 3^{rd} year | 4^{th} year | 5^{th} year |
| Conventional | 48 | 839.85 ± 112.98 | 732.80 ± 104.75 | 620.32 ± 90.75 | 610.53 ± 110.80 | 580.37 ± 85.20 | 520.82 ± 60.75 |
| treatment | | | | | | | |
| Conventional | 52 | 814.85 ± 118.76 | 740.32 ± 160.28 | 669.41 ± 140.55 | 640.64 ± 106.31 | 610.32 ± 78.50 | 580.41 ± 70.82 |
| treatment + | | | | | | | |
| Test | | | | | | | |
| formulation | | | | | | | |

Normal range: 710-1520 (mg/dl)

TABLE 4
Effect of test formulation on immunologic markers among HIV infected patients

| | No. | | | | | | |
|---|---|---|---|---|---|---|---|
| Treatment | of | IgM (mg/dI) | | | | | |
| group | cases | Initial | 1^{st} year | 2^{nd} year | 3^{rd} year | 4^{th} year | 5^{th} year |
| Conventional | 48 | 190.50 ± 61.30 | 160.55 ± 60.32 | 130.64 ± 22.80 | 108.51 ± 29.55 | 88.37 ± 27.20 | 34.75 ± 10.85 |
| treatment | | | | | | | |
| Conventional | 52 | 200.50 ± 88.50 | 180.85 ± 24.50 | 160.32 ± 25.35 | 140.50 ± 35.12 | 120.62 ± 38.40 | 69.52 ± 20.81 |
| treatment + | | | | | | | |
| Test | | | | | | | |
| formulation | | | | | | | |

Normal range: 40-250(mg/dl)

TABLE 5
Effect of test formulation on immunologic markers among HIV infected patients

| | No. | | | | | | |
|---|---|---|---|---|---|---|---|
| Treatment | of | IgA (mg/dI) | | | | | |
| group | cases | Initial | 1^{st} year | 2^{nd} year | 3^{rd} year | 4^{th} year | 5^{th} year |
| Conventional | 48 | 205.37 ± 48.30 | 216.85 ± 40.32 | 150.60 ± 48.32 | 120.35 ± 63.75 5 | 110.27 ± 34.80 | 270.25 ± 16.80 |
| treatment | | | | | | | |
| Conventional | 52 | 199.52 ± 22.80 | 167.40 ± 20.85 | 168.10 ± 17.45 | 140.53 ± 19.85 | 111.74 ± 29.32 | 98.50 ± 24.43 |
| treatment + | | | | | | | |
| Test | | | | | | | |
| formulation | | | | | | | |

Normal range: 90-310 (mg/dl)

TABLE 6
Level of depression in HIV infected patients following test formulation

| | No. | | | | | | |
|---|---|---|---|---|---|---|---|
| Treatment | of | IgA (mg/dI) | | | | | |
| group | cases | Initial | 1^{st} year | 2^{nd} year | 3^{rd} year | 4^{th} year | 5^{th} year |
| Conventional | 48 | 26.70 ± 4.85 | 27.40 ± 6.71 | 28.20 ± 4.85 | 29.42 ± 6.20 | 29.42 ± 6.20 | 31.10 ± 5.32 |
| treatment | | | | | | | |
| Conventional | 52 | 27.40 ± 6.40 | 26.20 ± 5.82 | 24.30 ± 4.85 | 24.30 ± 4.85 | 24.30 ± 4.85 | 22.63 ± 4.96 |
| treatment + | | | | | | | |
| Test | | | | | | | |
| formulation | | | | | | | |

In other words disease process is significantly slowed down under influence of test formulation treatment.

It is to be noted that the present invention is susceptible to modifications, adaptations and changes by those skilled in the art. Such variant embodiments employing the concepts and features of this invention are intended to be within the scope of the present invention, which is further set forth under the following claims.

We claim:

1. A novel herbal formulation for the modulation of immune system of HIV infected patients and a process of preparation thereof, comprising:
i. preparing a hydro-methanolic extract of at least one plant selected from Hippophae rhamnoides, Convolvulus pluricaulis, Withania somnifera, Ocimum sanctum, and Cynodon dactylon at 80-90° C., maintaining the pH of the solution between 6-7,
ii. separating the active compound chromatographically,
iii. subjecting the active compounds to the step of molecular characterization.

2. A novel herbal formulation as claimed in claim 1, wherein the said hydro-methanolic extract comprises of water and methanol in the ratio 50:50.

3. A novel herbal composition as claimed in claim 1, wherein different parts of the plants are used for preparing the extract as given below:

| | |
|---|---|
| 1. Hippophae rhamnoides (Badriphal) | fruits |
| 2. Withania somnifera (Ashwagandha | root |
| 3. Convolvulus pluricaulis (Shankhapushpi) | whole plant |
| 4. Cynodon dactylon (Durva) | whole plant |
| 5. Ocimum sanctum (Tulsi) | leaves |

4. A novel herbal formulation as claimed in claim 1, wherein the said plant extract are present in the herbal formulation in the following doses:

| | |
|---|---|
| 1. Hippophae rhamnoides (Badriphal) | 150-400 mg/day |
| 2. Withania somnifera (Ashwagandha) | 100-400 mg/day |
| 3. Convolvulus pluricaulis (Shankhapushpi) | 100-300 mg/day |
| 4. Cynodon dactylon (Durva) | 125-400 mg/day |
| 5. Ocimum sanctum (Tulsi) | 75-300 mg/day |

5. A novel herbal formulation as claimed in claim 4, wherein the said plant extract are preferably present in the herbal formulation in the following doses:

| | |
|---|---|
| 1. Hippophae rhamnoides (Badriphal) | 250 mg/day |
| 2. Withania somnifera (Ashwagandha) | 150 mg/day |
| 3. Convolvulus pluricaulis (Shankhapushpi) | 175 mg/day |
| 4. Cynodon dactylon (Durva) | 225 mg/day |
| 5. Ocimum sanctum (Tulsi) | 150 mg/day |

6. A novel herbal formulation as claimed in claim 1, comprising of hydro-methanolic extract of Hippophae rhamnoides, Withania somnifera, Convolvulus pluricaulis, Ocimum sanctum, Cynodon dactylon in effective doses having potentiality to enhance cell mediated immunity as well as humoral immunity among HIV patients.

7. A novel herbal formulation as claimed in claim 1, comprising of hydro-methanolic extract of Hippophae rhamnoides, Withania somnifera, Convolvulus pluricaulis, Ocimum sanctum, Cynodon dactylon in effective doses having neuro-protective, anti-anxiety and anti-depressant effect improving sleep pattern of HIV patients.

8. A novel herbal formulation as claimed in claim 1 comprising of hydro-methanolic extract of Hippophae rhamnoides, Withania somnifera, Convolvulus pluricaulis, Ocimum sanctum, Cynodon dactylon having hepato-protective and reno-protective property.

9. A novel herbal formulation as claimed in claim 1, comprising of hydro-methanolic extract of Hippophae rhamnoides, Withania somnifera, Convolvulus pluricaulis, Ocimum sanctum, Cynodon dactylon having homocystein lowering effects and anti-inflammatory activity reducing interleukins and TNF-α concentrations in HIV patients thereby preventing the HIV infected patients from neurodegenerative and cardiovascular complications.

10. A novel herbal formulation as claimed in claim 1, comprising of extract of Hippophae rhamnoides, Withania somnifera, Convolvulus pluricaulis, Ocimum sanctum, Cynodon dactylon having potentiality to prevent neutropenia and thrombocytopenia among HIV infected patients.

11. A novel herbal formulation as claimed in claim 1, comprising of hydro-methanolic extract of Hippophae rhamnoides, Withania somnifera, Convolvulus pluricaulis, Ocimum sanctum, Cynodon dactylon in effective doses having property to modulate immune profile in HIV infected patients.

12. A novel herbal formulation as claimed in claim 1, comprising of hydro-methanolic extract of Hippophae rhamnoides, Withania somnifera, Convolvulus pluricaulis, Ocimum sanctum, Cynodon dactylon in effective doses having property to produce good symptomatic improvement in HIV infected patients.

13. A novel herbal formulation as claimed in claim 1, comprising of hydro-methanolic extract of Hippophae rhamnoides, Withania somnifera, Convolvulus pluricaulis, Ocimum sanctum, Cynodon dactylon in effective doses having property to decrease the severity of symptoms such as diarrhea, fatigue, anorexia, cough and fever.

14. A novel herbal formulation as claimed in claim 1, comprising of hydro-methanolic extract of Hippophae rhamnoides, Withania somnifera, Convolvulus pluricaulis, Ocimum sanctum, Cynodon dactylon in effective doses having potentiality to decrease the mean viral load with increase in the mean CD4 cell count.

15. A novel herbal formulation as claimed in claim 1, comprising of hydro-methanolic extract of Hippophae rhamnoides, Withania somnifera, Convolvulus pluricaulis, Ocimum sanctum, Cynodon dactylon in effective doses having good immuno-modulatory effect with good potential as a therapeutic agent in the management of HIV infection.

16. A novel herbal formulation as claimed in claim 1, comprising of hydro-methanolic extract of Hippophae rhamnoides, Withania somnifera, Convolvulus pluricaulis, Ocimum sanctum, Cynodon dactylon in effective doses having property to produce its potent anti-oxidant activity the drug has shown immuno-stimulatory role and prevents the mitochondrial damage.

17. A novel herbal formulation as claimed in claim 1, comprising of hydro-methanolic extract of Hippophae rhamnoides, Withania somnifera, Convolvulus pluricaulis, Ocimum sanctum, Cynodon dactylon in effective doses having potentiality to prevent the HIV patients from opportunistic infectious, prevents neurodegeneration, improved quality of life and enhances longevity of HIV infected patients.
